SEQUENCE LISTING (88 sequences)

SEQ ID NO: 1 - protein, 8 aa, Artificial Sequence
  Description of Artificial Sequence Synthetic peptide
GYTFTDYN

SEQ ID NO: 2 - protein, 8 aa, Artificial Sequence
  Description of Artificial Sequence Synthetic peptide
INPNNGGT

SEQ ID NO: 3 - protein, 15 aa, Artificial Sequence
  Description of Artificial Sequence Synthetic peptide
ARDPDGYYPFYGMDY

SEQ ID NO: 4 - protein, 6 aa, Artificial Sequence
  Description of Artificial Sequence Synthetic peptide
QNVGTN

SEQ ID NO: 5 - protein, 3 aa, Artificial Sequence
  Description of Artificial Sequence Synthetic peptide
GAS

SEQ ID NO: 6 - protein, 9 aa, Artificial Sequence
  Description of Artificial Sequence Synthetic peptide
AQYSSYPYT

SEQ ID NO: 7 - protein, 8 aa, Artificial Sequence
  Description of Artificial Sequence Synthetic peptide
GFTFSNYA

SEQ ID NO: 8 - protein, 7 aa, Artificial Sequence
  Description of Artificial Sequence Synthetic peptide
ISSGGRI

SEQ ID NO: 9 - protein, 14 aa, Artificial Sequence
  Description of Artificial Sequence Synthetic peptide
TRGDDGYGSWYFDV

SEQ ID NO: 10 - protein, 10 aa, Artificial Sequence
  Description of Artificial Sequence Synthetic peptide
ESVDSYGNSF

SEQ ID NO: 11 - protein, 3 aa, Artificial Sequence
  Description of Artificial Sequence Synthetic peptide
RAS

SEQ ID NO: 12 - protein, 9 aa, Artificial Sequence
  Description of Artificial Sequence Synthetic peptide
QQNNEDPYT

SEQ ID NO: 13 - protein, 8 aa, Artificial Sequence
  Description of Artificial Sequence Synthetic peptide
GYTFTSYW

SEQ ID NO: 14 - protein, 8 aa, Artificial Sequence
  Description of Artificial Sequence Synthetic peptide
IDPNGGVT

SEQ ID NO: 15 - protein, 14 aa, Artificial Sequence
  Description of Artificial Sequence Synthetic peptide
TRENLYSKFAWFAY

SEQ ID NO: 16 - protein, 6 aa, Artificial Sequence
  Description of Artificial Sequence Synthetic peptide
QDVSTA

SEQ ID NO: 17 - protein, 3 aa, Artificial Sequence
  Description of Artificial Sequence Synthetic peptide
SAS

SEQ ID NO: 18 - protein, 9 aa, Artificial Sequence
  Description of Artificial Sequence Synthetic peptide
QQHYTTPWT

SEQ ID NO: 19 - protein, 8 aa, Artificial Sequence
  Description of Artificial Sequence Synthetic peptide
GYTFIRYY

SEQ ID NO: 20 - protein, 8 aa, Artificial Sequence
  Description of Artificial Sequence Synthetic peptide
INPSNGGT

SEQ ID NO: 21 - protein, 9 aa, Artificial Sequence
  Description of Artificial Sequence Synthetic peptide
TRLDGGFAY

SEQ ID NO: 22 - protein, 11 aa, Artificial Sequence
  Description of Artificial Sequence Synthetic peptide
QSIVHSDGKTY

SEQ ID NO: 23 - protein, 3 aa, Artificial Sequence
  Description of Artificial Sequence Synthetic peptide
KVS

SEQ ID NO: 24 - protein, 9 aa, Artificial Sequence
  Description of Artificial Sequence Synthetic peptide
FQGSHVPRT

SEQ ID NO: 25 - protein, 8 aa, Artificial Sequence
  Description of Artificial Sequence Synthetic peptide
GYTFTSYW

SEQ ID NO: 26 - protein, 8 aa, Artificial Sequence
  Description of Artificial Sequence Synthetic peptide
IHPYDSET

SEQ ID NO: 27 - protein, 7 aa, Artificial Sequence
  Description of Artificial Sequence Synthetic peptide
ADGSFVY

SEQ ID NO: 28 - protein, 11 aa, Artificial Sequence
  Description of Artificial Sequence Synthetic peptide
QSLLYSNGKTY

SEQ ID NO: 29 - protein, 3 aa, Artificial Sequence
  Description of Artificial Sequence Synthetic peptide
QVS

SEQ ID NO: 30 - protein, 9 aa, Artificial Sequence
  Description of Artificial Sequence Synthetic peptide
LQGTYYPRT

SEQ ID NO: 31 - protein, 8 aa, Artificial Sequence
  Description of Artificial Sequence Synthetic peptide
GFTFNTYA

SEQ ID NO: 32 - protein, 10 aa, Artificial Sequence
  Description of Artificial Sequence Synthetic peptide
IRSKSNNYAT

SEQ ID NO: 33 - protein, 14 aa, Artificial Sequence
  Description of Artificial Sequence Synthetic peptide
VRGGYDYDGLSMDY

SEQ ID NO: 34 - protein, 11 aa, Artificial Sequence
  Description of Artificial Sequence Synthetic peptide
KSLLHSNGNTY

SEQ ID NO: 35 - protein, 3 aa, Artificial Sequence
  Description of Artificial Sequence Synthetic peptide
RMS

SEQ ID NO: 36 - protein, 9 aa, Artificial Sequence
  Description of Artificial Sequence Synthetic peptide
MQHLEYPYT

SEQ ID NO: 37 - protein, 8 aa, Artificial Sequence
  Description of Artificial Sequence Synthetic peptide
GYSFTGYT

SEQ ID NO: 38 - protein, 8 aa, Artificial Sequence
  Description of Artificial Sequence Synthetic peptide
INPFNGGI

SEQ ID NO: 39 - protein, 13 aa, Artificial Sequence
  Description of Artificial Sequence Synthetic peptide
ASLYYDYSYAMDY

SEQ ID NO: 40 - protein, 6 aa, Artificial Sequence
  Description of Artificial Sequence Synthetic peptide
ENVGTY

SEQ ID NO: 41 - protein, 3 aa, Artificial Sequence
  Description of Artificial Sequence Synthetic peptide
GAS

SEQ ID NO: 42 - protein, 9 aa, Artificial Sequence
  Description of Artificial Sequence Synthetic peptide
GQSYSYPFT

SEQ ID NO: 43 - protein, 8 aa, Artificial Sequence
  Description of Artificial Sequence Synthetic peptide
GNTFTSYW

SEQ ID NO: 44 - protein, 8 aa, Artificial Sequence
  Description of Artificial Sequence Synthetic peptide
IHPGNSDT

SEQ ID NO: 45 - protein, 17 aa, Artificial Sequence
  Description of Artificial Sequence Synthetic peptide
TRGFRYYDGSYFYAMDY

SEQ ID NO: 46 - protein, 6 aa, Artificial Sequence
  Description of Artificial Sequence Synthetic peptide
ENIYSN

SEQ ID NO: 47 - protein, 3 aa, Artificial Sequence
  Description of Artificial Sequence Synthetic peptide
AAT

SEQ ID NO: 48 - protein, 9 aa, Artificial Sequence
  Description of Artificial Sequence Synthetic peptide
QHFYGTPYT

SEQ ID NO: 49 - DNA, 366 bp, Artificial Sequence
  Description of Artificial Sequence Synthetic polynucleotide
gaggtacagcttcaggagtctggacctgagctggtgaagcctggggcttcagtgaagatgtcctgcaaggcttctggatacacattcactgactacaacatgcactgggtgaaacagagccatggaaagagccttcagtggattggatatattaaccctaacaatggtggtactaggaacaaccagaagttcaagggcaaggccacattgactgtaaacaagtcctccagcacagcctacatggagctccgcagcctgacatcggaggactctgcagtctattactgtgcaagggatcctgatggttactacccattctatggtatggactattggggtcaagggaccttagtcaccgtctcctca

SEQ ID NO: 50 - protein, 122 aa, Artificial Sequence
  Description of Artificial Sequence Synthetic polypeptide
EVQLQESGPELVKPGASVKMSCKASGYTFTDYNMHWVKQSHGKSLQWIGYINPNNGGTRNNQKFKGKATLTVNKSSSTAYMELRSLTSEDSAVYYCARDPDGYYPFYGMDYWGQGTLVTVSS

SEQ ID NO: 51 - DNA, 323 bp, Artificial Sequence
  Description of Artificial Sequence Synthetic polynucleotide
gatattgtgatcactcagtctcaaaaattcatgtccacatcagtaggagagagggtcagcatcacctgcaaggccagtcagaatgtaggtactaatgtagcctggtatcagcagaaagcagggcagtctcttgaactgctgatctatggggcatccaaccggcacactggagtccctgatcacttcacaggcagtggatctgggacagatttcactctcaccatcaccaatgtgcagtctgaagacatgacaaattatttctgtgcgcaatatagcagctatccttatacgttcggatcggggaccaagctggaaatcaaacg

SEQ ID NO: 52 - protein, 107 aa, Artificial Sequence
  Description of Artificial Sequence Synthetic polypeptide
DIVITQSQKFMSTSVGERVSITCKASQNVGTNVAWYQQKAGQSLELLIYGASNRHTGVPDHFTGSGSGTDFTLTITNVQSEDMTNYFCAQYSSYPYTFGSGTKLEIK

SEQ ID NO: 53 - DNA, 360 bp, Artificial Sequence
  Description of Artificial Sequence Synthetic polynucleotide
gaggtgaagctggtggagtctgggggaggcttagtgaagcctggagggtccctgaaactctcctgtgtagcctctggattcactttcagtaactatgccatgtcttgggttcgtcagactccagaaaagaggctggagtgggtcgcatcaattagtagtggtggtagaatctactatccagacagtgtgaagggccgattcaccatctccagagataatgccaggaacatcctgtacctgcaaatgcgcagtctgaggtctgaggacacggccatgtattactgtacaagaggcgatgatggttacgggagctggtacttcgatgtctggggcgcagggacctcagtcaccgtctcctca

SEQ ID NO: 54 - protein, 120 aa, Artificial Sequence
  Description of Artificial Sequence Synthetic polypeptide
EVKLVESGGGLVKPGGSLKLSCVASGFTFSNYAMSWVRQTPEKRLEWVASISSGGRIYYPDSVKGRFTISRDNARNILYLQMRSLRSEDTAMYYCTRGDDGYGSWYFDVWGAGTSVTVSS

SEQ ID NO: 55 - DNA, 335 bp, Artificial Sequence
  Description of Artificial Sequence Synthetic polynucleotide
gacattcagatgatgcagtctccagcttctttggctgtgtctctgaggcagagggccaccatatcctgcagagccagtgaaagtgttgatagttatggcaatagttttatgcactggtaccagcagaaaccaggacagccacccaaactcctcatctatcgtgcatccaacctagaatctggggtccctgccaggttcagtggcagtgggtctaggacagacttcaccctcaccattgatcctgtggaggctgatgatgctgcaacctactactgtcagcaaaataatgaggatccgtatacgttcggaggggggaccaagctggaaataaaacg

SEQ ID NO: 56 - protein, 111 aa, Artificial Sequence
  Description of Artificial Sequence Synthetic polypeptide
DIQMMQSPASLAVSLRQRATISCRASESVDSYGNSFMHWYQQKPGQPPKLLIYRASNLESGVPARFSGSGSRTDFTLTIDPVEADDAATYYCQQNNEDPYTFGGGTKLEIK

SEQ ID NO: 57 - DNA, 363 bp, Artificial Sequence
  Description of Artificial Sequence Synthetic polynucleotide
caggtgcagctgaagcagtctggggctgagcttgtgaagcctggggcttcagtaaagctgtcctgcaaggcttctggctacaccttcaccagctactggatgcactgggtaaaacagaggcctggacgaggccttgagtggattggaaagattgatcctaatggtggtgtaactacgtacaatgagaagttcaggagcaaggccacactgactgtagacaaaccctccagcacagcctacatgcagctcagcagtctgacatctgaggactctgcggtctattactgtacaagagaaaacttatatagtaaattcgcctggtttgcttactggggccaagggactctggtcactgtctctgca

SEQ ID NO: 58 - protein, 121 aa, Artificial Sequence
  Description of Artificial Sequence Synthetic polypeptide
QVQLKQSGAELVKPGASVKLSCKASGYTFTSYWMHWVKQRPGRGLEWIGKIDPNGGVTTYNEKFRSKATLTVDKPSSTAYMQLSSLTSEDSAVYYCTRENLYSKFAWFAYWGQGTLVTVSA

SEQ ID NO: 59 - DNA, 322 bp, Artificial Sequence
  Description of Artificial Sequence Synthetic polynucleotide
gatattcagatgaaccagtctcacaaattcatgtccacatcggtaggagacagggtctccatcacctgcaaggccagtcaggatgtgagtactgctgtagcctggtatcaacagaaaccaggacaatctccaaaactactgatttactcggcatcctaccggtacactggagtccctggtcgtttcactggcagtggatctgggacggatttcactttcaccatcagcagtgtgcaggctgaagacctggcaatttattactgtcagcaacattatactactccgtggacgttcggtggaggcaccaagctggaaatcaaac

SEQ ID NO: 60 - protein, 107 aa, Artificial Sequence
  Description of Artificial Sequence Synthetic polypeptide
DIQMNQSHKFMSTSVGDRVSITCKASQDVSTAVAWYQQKPGQSPKLLIYSASYRYTGVPGRFTGSGSGTDFTFTISSVQAEDLAIYYCQQHYTTPWTFGGGTKLEIK

SEQ ID NO: 61 - DNA, 348 bp, Artificial Sequence
  Description of Artificial Sequence Synthetic polynucleotide
gaggtgcagcttcagcagtctggggctgaactggtgaagcctggggcttcagtgaagttgtcctgcaaggcttctggctacaccttcattaggtactatatgtactgggtgaaacagaggcctggacaaggccttgagtggattggagagattaatcctagcaatggtggtactaacttcaatgagaagttcaagaacaaggccacactgactgtagacaaatcctccaggacagtatatatgcaaattagtagcctgacatctgaggattctgcggcctattactgtacaagattagacggagggtttgcttactggggccaagggactctggtcactgtctctgca

SEQ ID NO: 62 - protein, 116 aa, Artificial Sequence
  Description of Artificial Sequence Synthetic polypeptide
EVQLQQSGAELVKPGASVKLSCKASGYTFIRYYMYWVKQRPGQGLEWIGEINPSNGGTNFNEKFKNKATLTVDKSSRTVYMQISSLTSEDSAAYYCTRLDGGFAYWGQGTLVTVSA

SEQ ID NO: 63 - DNA, 337 bp, Artificial Sequence
  Description of Artificial Sequence Synthetic polynucleotide
gatattgtgatgacccagactccactctccctgcctgtcagtcttggagatcaagcctccatctcttgcagatctagtcagagcattgtacatagtgatggaaaaacctatttagaatggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggaatttattactgctttcaaggttcacatgttcctcggacgttcggtggaggcaccaagctggaaataaaac

SEQ ID NO: 64 - protein, 112 aa, Artificial Sequence
  Description of Artificial Sequence Synthetic polypeptide
DIVMTQTPLSLPVSLGDQASISCRSSQSIVHSDGKTYLEWYLQKPGQSPKLLIYKVSNRFSGVPDRFSGSGSGTDFTLRISRVEAEDLGIYYCFQGSHVPRTFGGGTKLEIK

SEQ ID NO: 65 - DNA, 342 bp, Artificial Sequence
  Description of Artificial Sequence Synthetic polynucleotide
gaggtccagctgcagcagtctggggctgagctggtgaggcctggggcttcagtgaaactgtcctgcaaggcttccggctacacattcaccagctactggatgcactggattaagcagaggcctgggcaaggtcttgagtggattggaacgattcatccttacgatagtgaaacacactacaatcaaaagttcaagggcaaggccacattgactgttgacaaatcctccaccacagcctacatgcagctcagcagcctgacatctgaggactctgcggtcttttattgtgccgatggttcctttgtttactggggccaagggactctggtcactgtctctgca

SEQ ID NO: 66 - protein, 114 aa, Artificial Sequence
  Description of Artificial Sequence Synthetic polypeptide
EVQLQQSGAELVRPGASVKLSCKASGYTFTSYWMHWIKQRPGQGLEWIGTIHPYDSETHYNQKFKGKATLTVDKSSTTAYMQLSSLTSEDSAVFYCADGSFVYWGQGTLVTVSA

SEQ ID NO: 67 - DNA, 337 bp, Artificial Sequence
  Description of Artificial Sequence Synthetic polynucleotide
gatattgtgatgacccagtctccactgtctttgtcggttaccattggacaaccagcctccatatcttgcaagtcaagtcagagcctcttatatagtaatggaaagacatatttgaattggttacaactgaggcctggccagtctccaaagcgcctaatctctcaggtgtccaaactggaccctggcatccctgacaggttcagtggcagtggatcagaaacagattttacacttaaaatcagcagagtggaggctgaagatttgggattttattactgcttgcaaggtacatattatcctcggacgttcggtggaggcaccaagctggaaatcaaac

SEQ ID NO: 68 - protein, 112 aa, Artificial Sequence
  Description of Artificial Sequence Synthetic polypeptide
DIVMTQSPLSLSVTIGQPASISCKSSQSLLYSNGKTYLNWLQLRPGQSPKRLISQVSKLDPGIPDRFSGSGSETDFTLKISRVEAEDLGFYYCLQGTYYPRTFGGGTKLEIK

SEQ ID NO: 69 - DNA, 369 bp, Artificial Sequence
  Description of Artificial Sequence Synthetic polynucleotide
cgaggtgatgctggtgagtctggtggaggattggtgcagcctaaaggatcattgaaactctcatgtgccgcctctggtttcaccttcaatacctatgccatgcactgggtctgccaggctccaggaaagggtttggaatgggttgctcgcataagaagtaaaagtaataattatgcaacatattatgccgattcagtgaaagacagattcaccatctccagagatgattcacaaagcatgctctatctgcaaatgaacaacctgaaaactgaggacacagccatgtattactgtgtgagaggggggtatgattacgacggcctttctatggactactggggtcaaggaacctcagtcaccgtctcctca

SEQ ID NO: 70 - protein, 123 aa, Artificial Sequence
  Description of Artificial Sequence Synthetic polypeptide
RGDAGESGGGLVQPKGSLKLSCAASGFTFNTYAMHWVCQAPGKGLEWVARIRSKSNNYATYYADSVKDRFTISRDDSQSMLYLQMNNLKTEDTAMYYCVRGGYDYDGLSMDYWGQGTSVTVSS

SEQ ID NO: 71 - DNA, 338 bp, Artificial Sequence
  Description of Artificial Sequence Synthetic polynucleotide
gacattgtgatgactcagtctgcaccctctgtacctgtcactcctggagagtcagtgtccatctcctgcaggtctagtaagagtctcctgcatagtaatggcaacacttacttgtattggttcctgcagaggccaggccagtctcctcagctcctgatatatcggatgtccgaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaatcaaacg

SEQ ID NO: 72 - protein, 112 aa, Artificial Sequence
  Description of Artificial Sequence Synthetic polypeptide
DIVMTQSAPSVPVTPGESVSISCRSSKSLLHSNGNTYLYWFLQRPGQSPQLLIYRMSDLASGVPDRFSGSGSGTAFTLRISRVEAEDVGVYYCMQHLEYPYTFGGGTKLEIK

SEQ ID NO: 73 - DNA, 360 bp, Artificial Sequence
  Description of Artificial Sequence Synthetic polynucleotide
caggtccaactgcagcagtctggacctgagctggtgaagcctggagcttcaatgaagatatcctgcaaggcttctggttactcattcactggctacaccatgaactgggtgaagcagagccatggaaagaaccttgagtggattggacttattaatcctttcaatggtggtattacctacaaccagaagttcaagggcaaggccacattaactgtagacacgtcatccagcacagcctacatggagctcctcagtctgacatctgaggactctgcagtctattactgtgcaagcctctactatgattactcctatgctatggactactggggtcaaggaacctcagtcaccgtctcctca

SEQ ID NO: 74 - protein, 120 aa, Artificial Sequence
  Description of Artificial Sequence Synthetic polypeptide
QVQLQQSGPELVKPGASMKISCKASGYSFTGYTMNWVKQSHGKNLEWIGLINPFNGGITYNQKFKGKATLTVDTSSSTAYMELLSLTSEDSAVYYCASLYYDYSYAMDYWGQGTSVTVSS

SEQ ID NO: 75 - DNA, 322 bp, Artificial Sequence
  Description of Artificial Sequence Synthetic polynucleotide
gacattcagatgatgcagtctcccaaatccatgtccatgtcagtaggagagagggtcaccttgagctgcaaggccagtgagaatgtgggtacttatgtatcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattattactgtggacagagttacagctatccattcacgttcggcacggggacaaagttggaaataaaac

SEQ ID NO: 76 - protein, 107 aa, Artificial Sequence
  Description of Artificial Sequence Synthetic polypeptide
DIQMMQSPKSMSMSVGERVTLSCKASENVGTYVSWYQQKPEQSPKLLIYGASNRYTGVPDRFTGSGSATDFTLTISSVQAEDLADYYCGQSYSYPFTFGTGTKLEIK

SEQ ID NO: 77 - DNA, 372 bp, Artificial Sequence
  Description of Artificial Sequence Synthetic polynucleotide
caggttcagctgcagcagtctgggactgtgctggcaaggcctggggcttcagtgaagatgtcctgcaagacttctggcaacacatttaccagctactggatgcactggataaaacagaggcctggacagggtctggaatggatagggactattcatcctggaaatagtgatactacctacaaccagaagttcaagggcaaggccaaactgactgcagtcacatccgccagcactgcctacatggagctcagcagcctgacaaatgaggactctgcggtctattactgtacaagaggatttcgttactacgatggtagctacttctatgctatggactactggggtcaaggaacctcagtcaccgtctcctca

SEQ ID NO: 78 - protein, 124 aa, Artificial Sequence
  Description of Artificial Sequence Synthetic polypeptide
QVQLQQSGTVLARPGASVKMSCKTSGNTFTSYWMHWIKQRPGQGLEWIGTIHPGNSDTTYNQKFKGKAKLTAVTSASTAYMELSSLTNEDSAVYYCTRGFRYYDGSYFYAMDYWGQGTSVTVSS

SEQ ID NO: 79 - DNA, 323 bp, Artificial Sequence
  Description of Artificial Sequence Synthetic polynucleotide
gatattgtgatgactcagtctccagcctccctatctgcatctgtgggagaaactgtcaccatcacatgtcgagcaagtgagaatatttacagtaatttagcatggtatcagcagaaacagggaaaatctcctcagctcctggtctatgctgcaacaaatttagcagatggtgtgccatcaaggttcagtggcagtggatcaggcacacagttttctctgaagatcaatagcctgcagcctgaagattttgggagttattactgtcaacatttttatggtactccgtatacgttcggatcggggaccaagctggaaatcaaacg

SEQ ID NO: 80 - protein, 107 aa, Artificial Sequence
  Description of Artificial Sequence Synthetic polypeptide
DIVMTQSPASLSASVGETVTITCRASENIYSNLAWYQQKQGKSPQLLVYAATNLADGVPSRFSGSGSGTQFSLKINSLQPEDFGSYYCQHFYGTPYTFGSGTKLEIK

SEQ ID NO: 81 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 82 - protein, 8 aa, Artificial Sequence
  Description of Artificial Sequence Synthetic peptide
GXTFTSYW

SEQ ID NO: 83 - protein, 8 aa, Artificial Sequence
  Description of Artificial Sequence Synthetic peptide
INPXNGGX

SEQ ID NO: 84 - protein, 8 aa, Artificial Sequence
  Description of Artificial Sequence Synthetic peptide
SQPLPGER

SEQ ID NO: 85 - protein, 7 aa, Homo sapiens
MPIMGSS

SEQ ID NO: 86 - protein, 11 aa, Homo sapiens
STGFCAACHGC

SEQ ID NO: 87 - protein, 29 aa, Homo sapiens
NNCGQPKEGKNHSQGCGEQVACLFEDVVP

SEQ ID NO: 88 - protein, 8 aa, Homo sapiens
CPDCSHAP

(19) United States
(12) Patent Application Publication
(10) Pub. No.: US 2014/0322236 A1
(43) Pub. Date: Oct. 30, 2014

(54) ANTI-HUMAN ADORA2A ANTIBODIES

(71) Applicant: SDIX, LLC, Rockville, MD (US)

(72) Inventors: Ross S. CHAMBERS, Hockessin, DE (US); Michael C. BROWN, North East, MD (US); Dale V. ONISK, Conowingo, MD (US); L. Joe STAFFORD, Newark, DE (US); Fenglin YIN, Naperville, IL (US)

(73) Assignee: SDIX, LLC, Rockville, MD (US)

(21) Appl. No.: 14/217,837

(22) Filed: Mar. 18, 2014

Related U.S. Application Data
(60) Provisional application No. 61/801,080, filed on Mar. 15, 2013.

(51) Int. Cl. C07K 16/28

(52) U.S. Cl. CPC C07K 16/28
USPC 424/172.1; 530/389.1; 530/387.3; 536/23.53; 435/320.1; 435/69.6

(57) ABSTRACT

The present invention provides novel antibodies to human ADORA2A.

### BACKGROUND OF THE INVENTION

ADORA2A is an intensely studied G-protein coupled receptor (GPCR) and has relevance to a wide range of therapeutic indications including diseases of the inflammatory and immune systems, neurological diseases, and endocrine disorders. Anti-ADORA2A antibodies are commercially available for research, but none are able to recognize the native extracellular epitopes, a critical requirement for therapeutic research applications (e.g. flow cytometry). For example, there are approximately 81 antibodies to ADORA2A, of which 7 are listed for flow cytometry. However, the putative flow cytometry ADORA2A antibodies have been shown not to work on native epitopes (i.e., permeabilization and fixing of the cells is required).

The extracellular regions of ADORA2A are composed of 4 discontinuous segments (N-terminal domain, extracellular loop (ECL) 1, ECL2, and ECL3), comprising a total of 56 amino acids. The extracellular regions are modified with disulfide bonds and N-linked glycosylation. Most importantly, the extracellular epitopes are extremely fragile and the protein must be maintained in the plasma membrane. These factors, as well as low expression levels make it difficult to generate antibodies against ADORA2A.

### SUMMARY OF THE INVENTION

In one aspect, the present invention provides an antibody comprising: a first heavy chain CDR comprising SEQ ID NO:1; a second heavy chain CDR comprising SEQ ID NO:2; a third heavy chain CDR comprising SEQ ID NO:3; a first light chain CDR comprising SEQ ID NO:4; a second light chain CDR comprising SEQ ID NO:5; and a third light chain CDR comprising SEQ ID NO:6, wherein the antibody binds to human ADORA2A.

In one aspect, the present invention provides an antibody comprising: a first heavy chain CDR comprising SEQ ID NO:7; a second heavy chain CDR comprising SEQ ID NO:8; a third heavy chain CDR comprising SEQ ID NO:9; a first light chain CDR comprising SEQ ID NO:10; a second light chain CDR comprising SEQ ID NO:11; and a third light chain CDR comprising SEQ ID NO:12, wherein said antibody binds to human ADORA2A.

In one aspect, the present invention provides an antibody comprising: a first heavy chain CDR comprising SEQ ID NO:13; a second heavy chain CDR comprising SEQ ID NO:14; a third heavy chain CDR comprising SEQ ID NO:15; a first light chain CDR comprising SEQ ID NO:16; a second light chain CDR comprising SEQ ID NO:17; and a third light chain CDR comprising SEQ ID NO:18, wherein said antibody binds to human ADORA2A.

In one aspect, the present invention provides an antibody comprising: a first heavy chain CDR comprising SEQ ID NO:19; a second heavy chain CDR comprising SEQ ID NO:20; a third heavy chain CDR comprising SEQ ID NO:21; a first light chain CDR comprising SEQ ID NO:22; a second light chain CDR comprising SEQ ID NO:23; and a third light chain CDR comprising SEQ ID NO:24, wherein said antibody binds to human ADORA2A.

In one aspect, the present invention provides an antibody comprising: a first heavy chain CDR comprising SEQ ID NO:25; a second heavy chain CDR comprising SEQ ID NO:26; a third heavy chain CDR comprising SEQ ID NO:27; a first light chain CDR comprising SEQ ID NO:28; a second light chain CDR comprising SEQ ID NO:29; and a third light chain CDR comprising SEQ ID NO:30, wherein said antibody binds to human ADORA2A.

In one aspect, the present invention provides an antibody comprising: a first heavy chain CDR comprising SEQ ID NO:31; a second heavy chain CDR comprising SEQ ID NO:32; a third heavy chain CDR comprising SEQ ID NO:33; a first light chain CDR comprising SEQ ID NO:34; a second light chain CDR comprising SEQ ID NO:35; and a third light chain CDR comprising SEQ ID NO:36, wherein said antibody binds to human ADORA2A.

In one aspect, the present invention provides an antibody comprising: a first heavy chain CDR comprising SEQ ID NO:37; a second heavy chain CDR comprising SEQ ID NO:38; a third heavy chain CDR comprising SEQ ID NO:39; a first light chain CDR comprising SEQ ID NO:40; a second light chain CDR comprising SEQ ID NO:41; and a third light chain CDR comprising SEQ ID NO:42, wherein said antibody binds to human ADORA2A.

In one aspect, the present invention provides an antibody comprising: a first heavy chain CDR comprising SEQ ID NO:43; a second heavy chain CDR comprising SEQ ID NO:44; a third heavy chain CDR comprising SEQ ID NO:45; a first light chain CDR comprising SEQ ID NO:46; a second light chain CDR comprising SEQ ID NO:47; and a third light chain CDR comprising SEQ ID NO:48, wherein said antibody binds to human ADORA2A.

### BRIEF DESCRIPTION OF THE DRAWINGS

These and other objects, features and advantages of the disclosure will be apparent from a consideration of the following non-limiting detailed description considered in conjunction with the drawing figures, in which:

FIG. 1 is an illustration of ADORA2A extracellular regions.

FIG. 2 is a series of images depicting the results of flow cytometry using ADORA2A antibodies on transfected HEK293 cells.

FIG. 3 is a chart illustrating CDR sequences of the VH and VK genes from the hybridomas prepared according to the disclosure set forth herein.

FIG. 4 provides the results of flow cytometry using a commercially available ADORA2A antibody on transfected HEK293 cells.

FIGS. 5-20 provide sequences of variable heavy chain and variable light chains of antibodies of the invention.

FIG. 21 provides a sequence of a human ADORA2A protein.

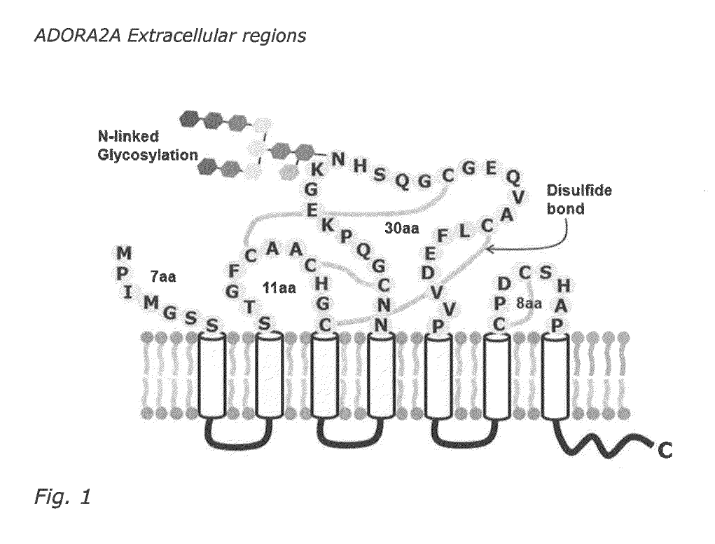

### DETAILED DESCRIPTION OF THE INVENTION

With reference to the accompanying drawings, various embodiments of the present invention are described more fully below. Some but not all embodiments of the present invention are shown. Indeed, various embodiments of the invention may be embodied in many different forms and should not be construed as limited to the embodiments expressly described. Like numbers refer to like elements throughout. The singular forms “a,” “an,” and “the” include the singular and plural unless the context clearly dictates otherwise.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

### I. OVERVIEW

The present invention is directed to novel antibodies to human ADORA2A. Surprisingly, unlike previously generated anti-ADORA2A antibodies, the anti-ADORA2A antibodies encompassed herein are able to bind to native extracellular ADORA2A epitopes in flow cytometry methods.

### II. DEFINITIONS

In order that the application may be more completely understood, several definitions are set forth below. Such definitions are meant to encompass grammatical equivalents.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233# designates a deletion of glutamic acid at position 233. Additionally, EDA233− or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L- (R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc fusion protein” or “immunoadhesin” herein is meant a protein comprising an Fc region, generally linked (optionally through a linker moiety, as described herein) to a different protein, such as a binding moiety to a target protein, as described herein).

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A wide number of suitable target antigens are described below.

By “target cell” as used herein is meant a cell that expresses a target antigen.

By “variable region” as used herein is meant the region of an immunoglobulin that comprises one or more Ig domains substantially encoded by any of the V.kappa., and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

### III. ADORA2A ANTIBODIES

The present invention provides novel antibodies to human ADORA2A. These antibodies, unlike previously generated anti-ADORA2A antibodies, are able to bind to the native extracellular ADORA2A epitopes in flow cytometry methods.

Antibodies that find use in the present invention can take on a number of formats as described herein, including traditional antibodies as well as antibody derivatives, fragments and mimetics, described below. In general, the term “antibody” includes any polypeptide that includes at least one constant domain, including, but not limited to, CH1, CH2, CH3 and CL.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. It should be understood that therapeutic antibodies can also comprise hybrids of isotypes and/or subclasses.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

Antibody Fragments

In one embodiment, the antibody is an antibody fragment. Of particular interest are antibodies that comprise Fc regions, Fc fusions, and the constant region of the heavy chain (CH1-hinge-CH2-CH3), again also including constant heavy region fusions.

Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference). The antibody fragments may be modified. For example, the molecules may be stabilized by the incorporation of disulphide bridges linking the VH and VL domains (Reiter et al., 1996, Nature Biotech. 14:1239-1245, entirely incorporated by reference).

Chimeric and Humanized Antibodies

In some embodiments, the scaffold components can be a mixture from different species. As such, if the protein is an antibody, such antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

In one embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

ADORA2A Antibodies and Variants

As used herein, the term “ADORA2A antibody” refers to any antibody that binds to ADORA2A, including antibodies comprising any of the sequences described herein and variants thereof.

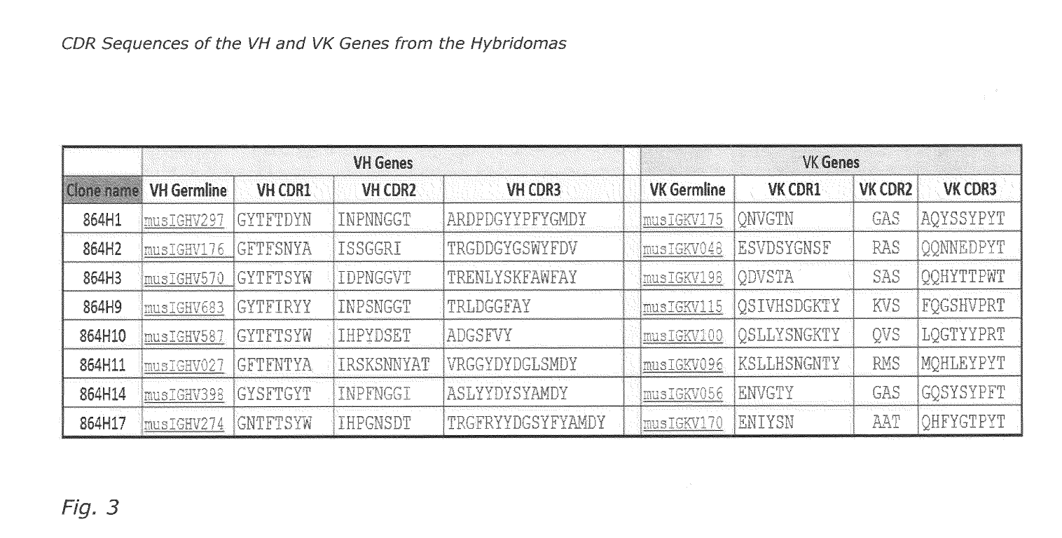

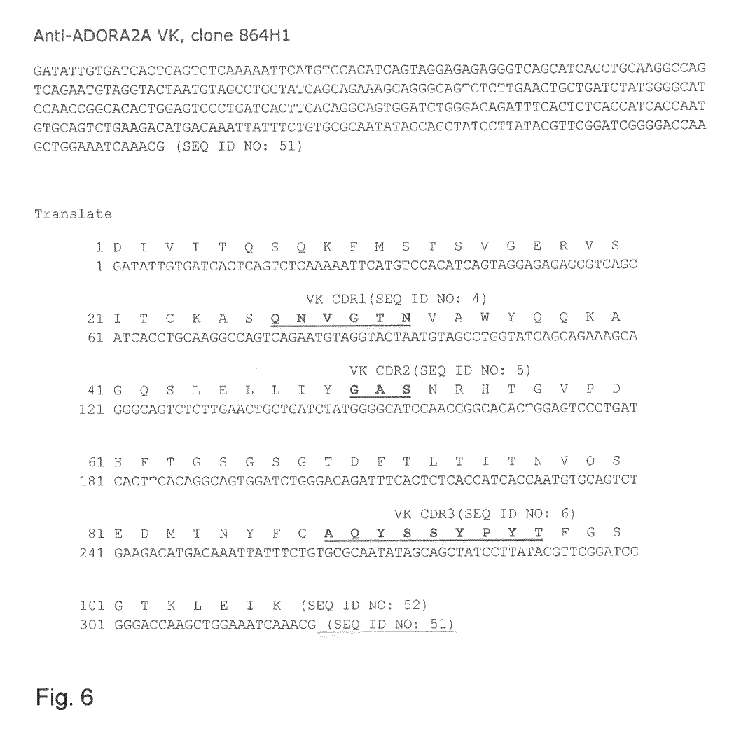

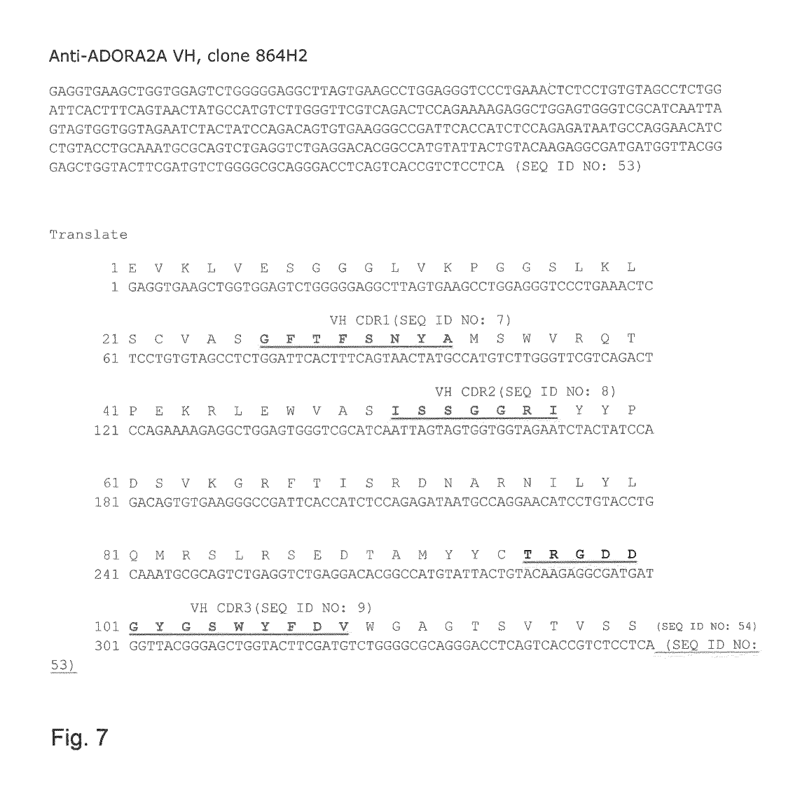

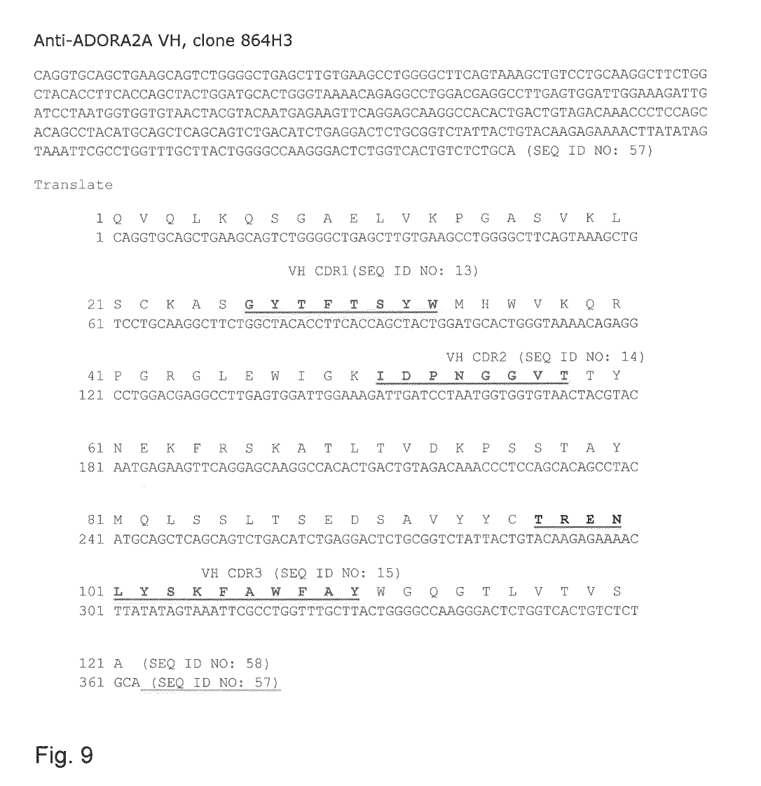

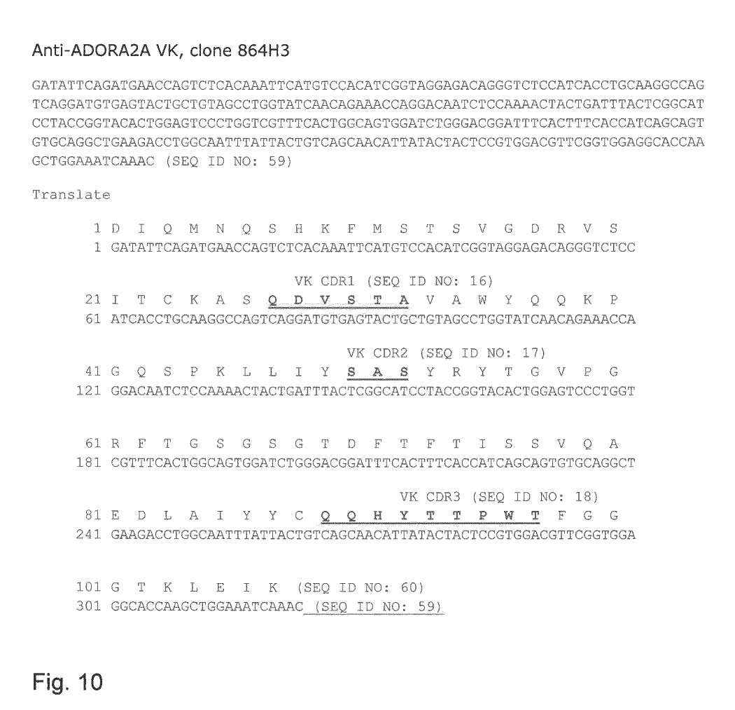

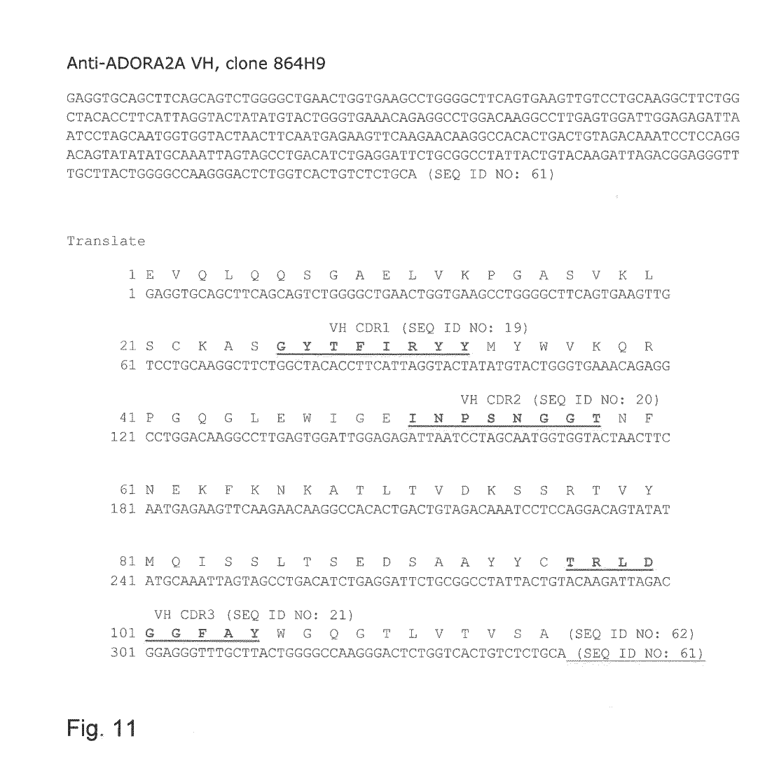

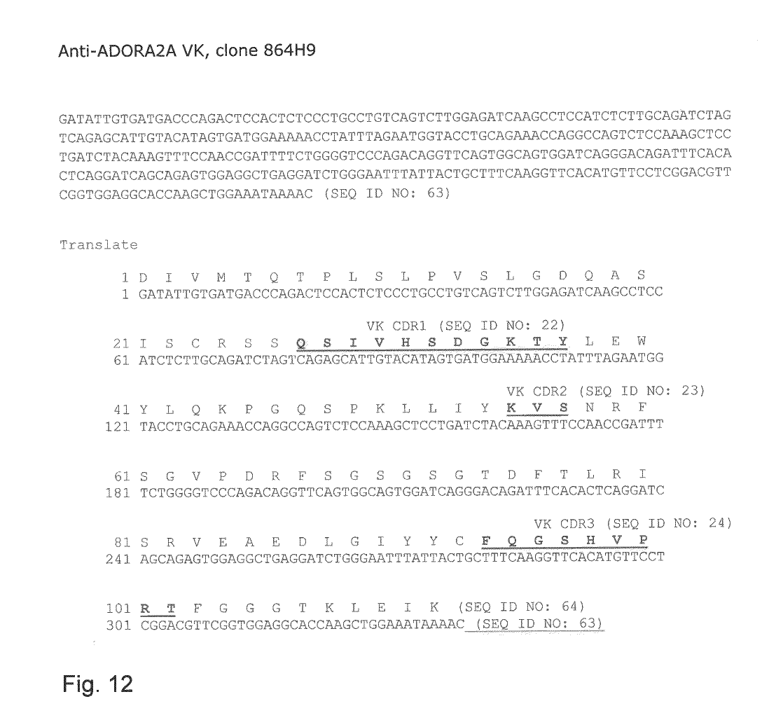

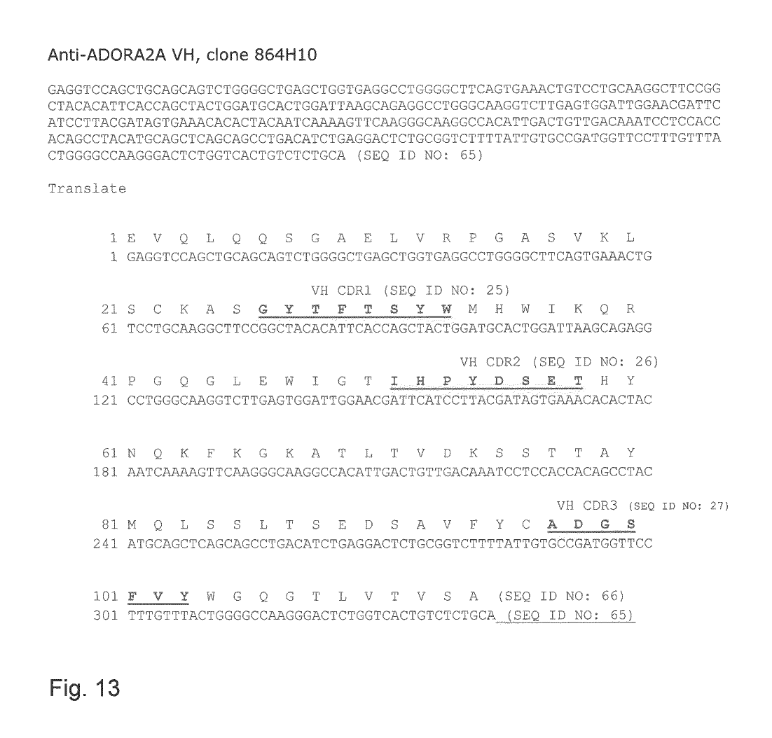

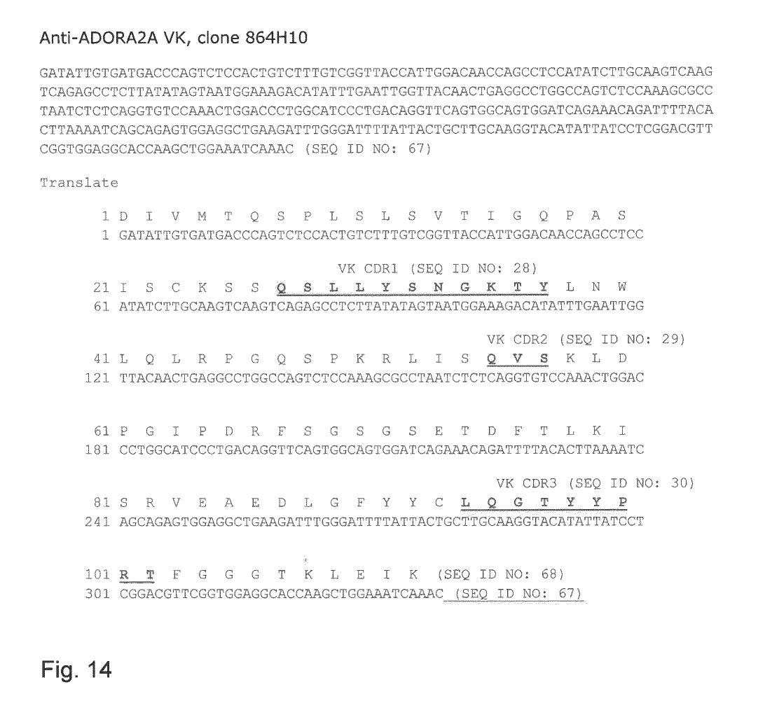

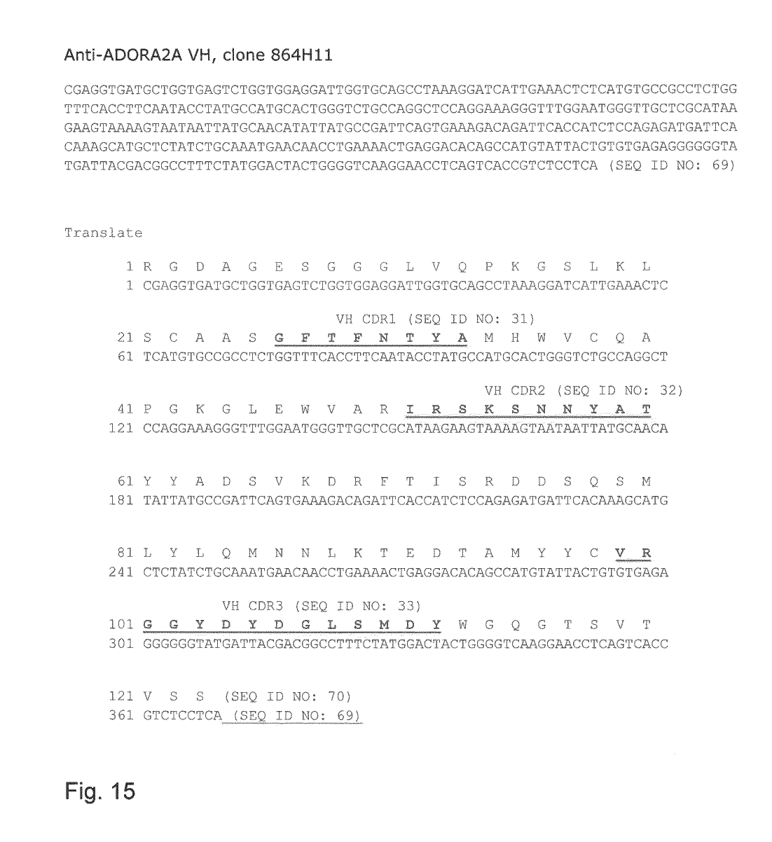

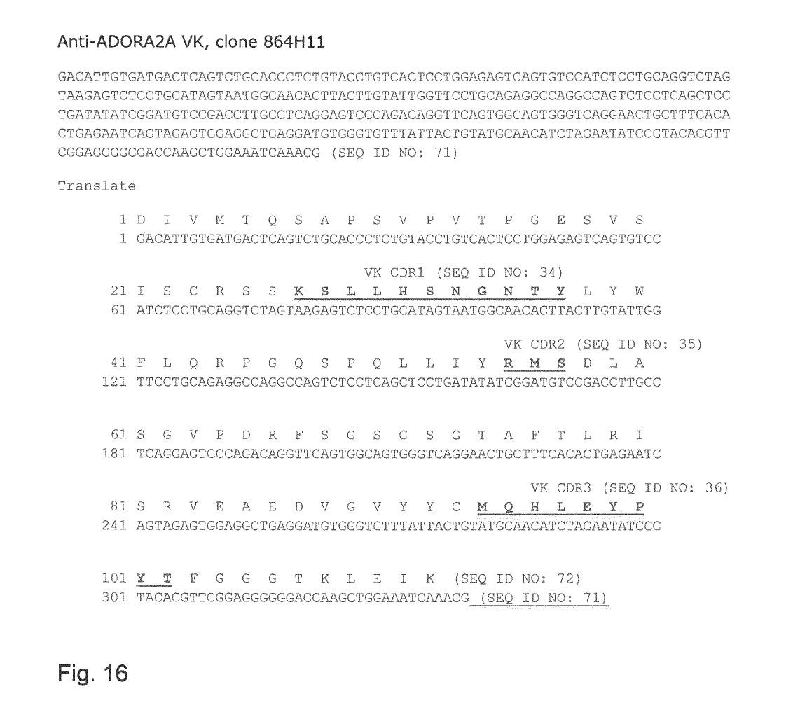

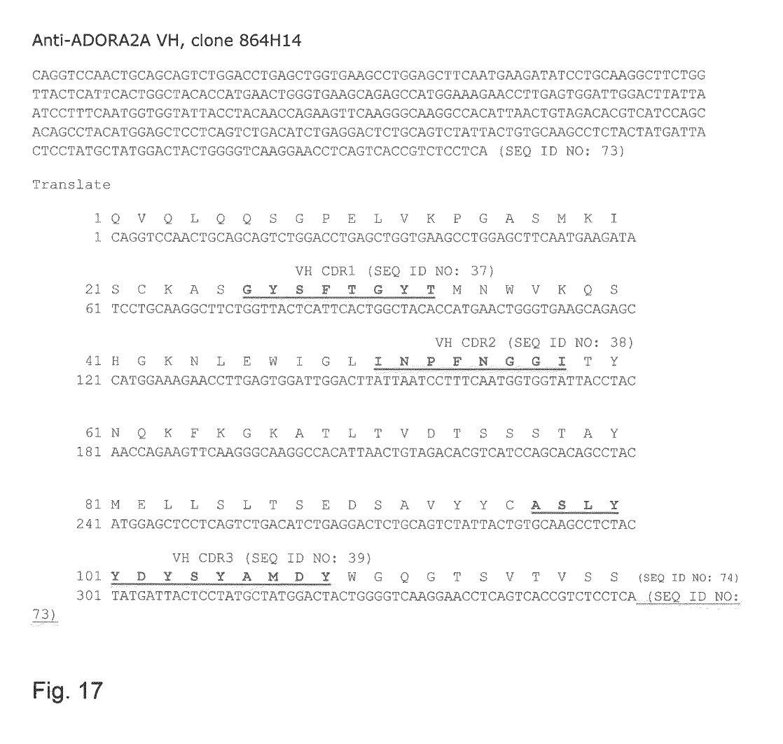

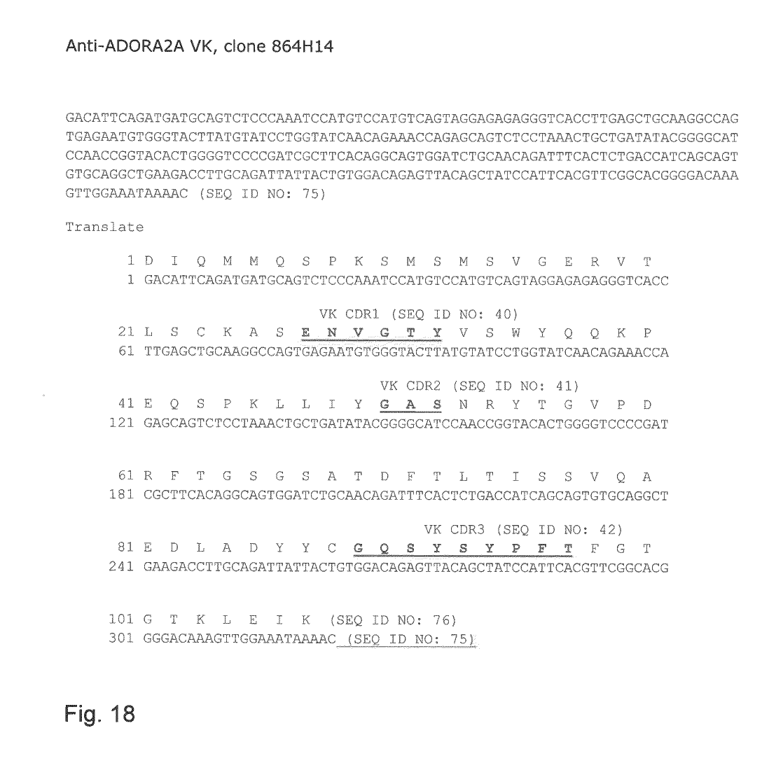

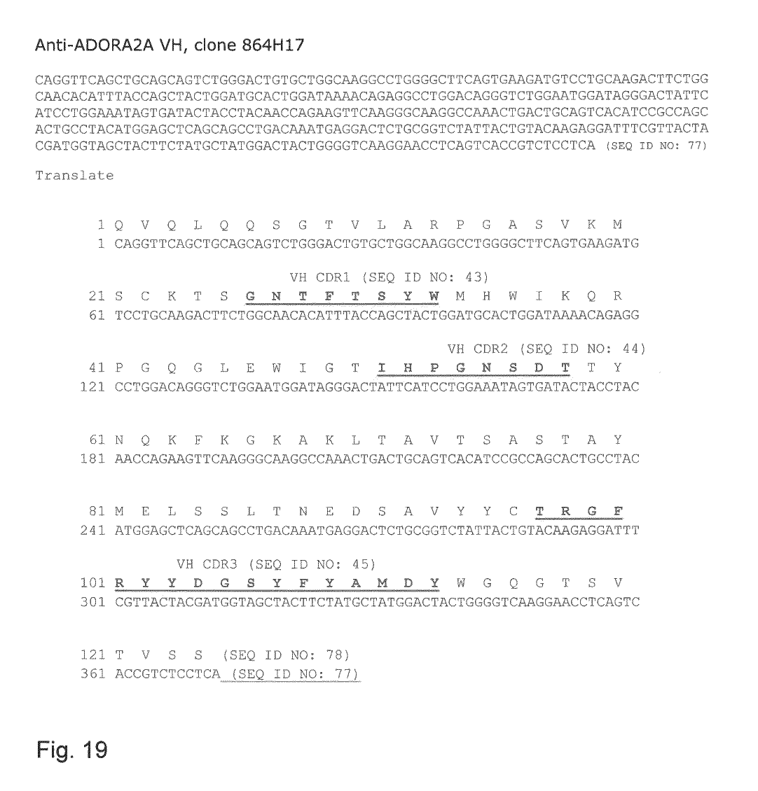

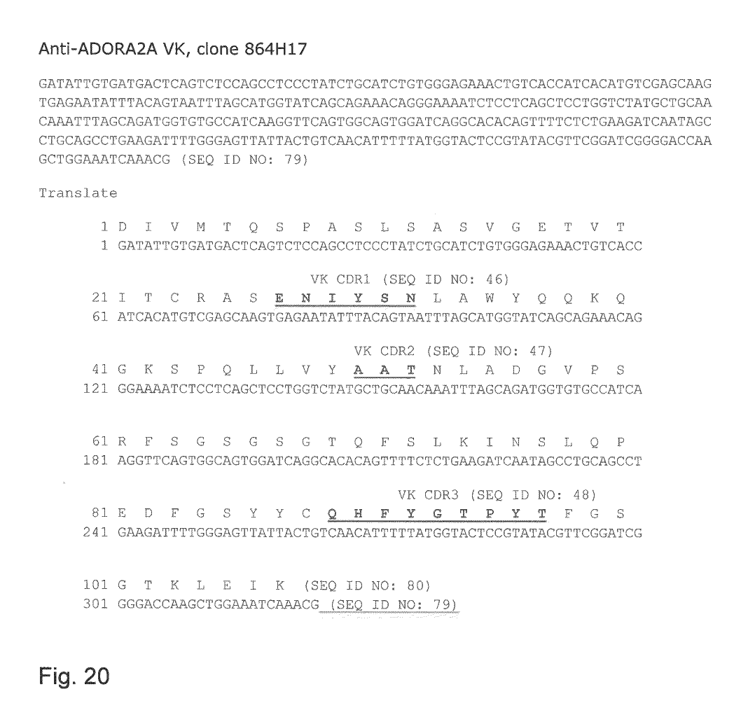

In certain aspects, ADORA2A antibodies of the invention include antibodies comprising CDR sequences as provided in FIG. 3. In further embodiments, ADORA2A antibodies of the invention comprise variants of the CDR sequences as provided in FIG. 3. In general, variants can include any number of modifications, as long as the function of the protein is still present, as described herein. That is, in the case of amino acid variants generated with the CDRs of any of the antibodies comprising sequences as provided in FIGS. 3 and 5-20, for example, the antibody should still specifically bind to both human ADORA2A. Similarly, if amino acid variants are generated with the Fc region, for example, the variant antibodies should maintain the required receptor binding functions for the particular application or indication of the antibody.

However, in general, from 1, 2, 3, 4, 5, 6, 7, 8, 9 or 10 amino acid substitutions are generally utilized as often the goal is to alter function with a minimal number of modifications. In some cases, there are from 1 to 5 modifications, with from 1-2, 1-3 and 1-4 also finding use in many embodiments.

In some embodiments, one or more amino acid modifications are made in one or more of the CDRs of the ADORA2A antibodies of the invention as provided in FIGS. 3 and 5-20. In general, only 1 or 2 or 3-amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR for a particular antibody can be independently and optionally combined with any other substitution.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Affinity maturation can be done to increase the binding affinity of the antibody for the antigen by at least about 10% to 50-100-150% or more, or from 1 to 5 fold as compared to the “parent” antibody. Preferred affinity matured antibodies will have nanomolar or even picomolar affinities for the target antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of antibodies 864H1, 864H2, 864H3, 864H9, 864H10, 864H11, 864H14, and 864H17, for which sequences of the CDRs and the heavy and light chains are provided in FIGS. 3 and 5-20. In addition, amino acid modifications can also independently and optionally be made in any region outside the CDRs, including framework and constant regions.

In some embodiments, the anti-ADORA2A antibodies of the invention are composed of a variant Fc domain. As is known in the art, the Fc region of an antibody interacts with a number of Fc receptors and ligands, imparting an array of important functional capabilities referred to as effector functions. These Fc receptors include, but are not limited to, (in humans) FcγRI (CD64) including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F 158, correlated to antibody-dependent cell cytotoxicity (ADCC)) and FcγRIIIb (including allotypes FcγRIIIb-NA1 and FcγRIIIb-NA2), FcRn (the neonatal receptor), C1q (complement protein involved in complement dependent cytotoxicity (CDC)) and FcRn (the neonatal receptor involved in serum half-life). Suitable modifications can be made at one or more positions as is generally outlined, for example in US 2004/013210, US 2005/0054832, US 2006/0024298, US 2006/0121032, US 2006/0235208, US 2007/0148170, U.S. Pat. No. 6,737,056, U.S. Pat. No. 7,670,600, U.S. Pat. No. 6,086,875 all of which are expressly incorporated by reference in their entirety, and in particular for specific amino acid substitutions that increase binding to Fc receptors.

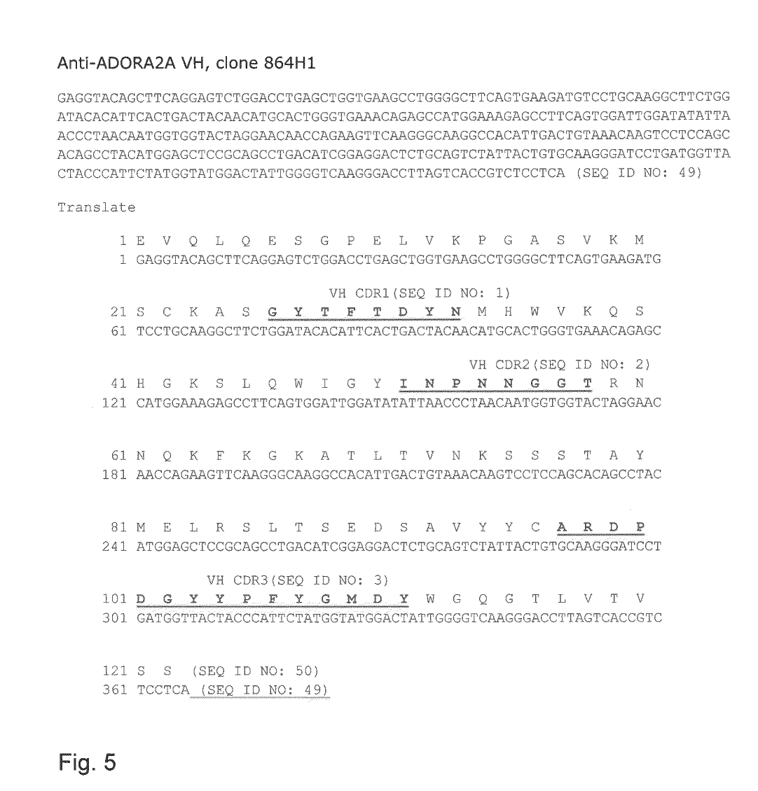

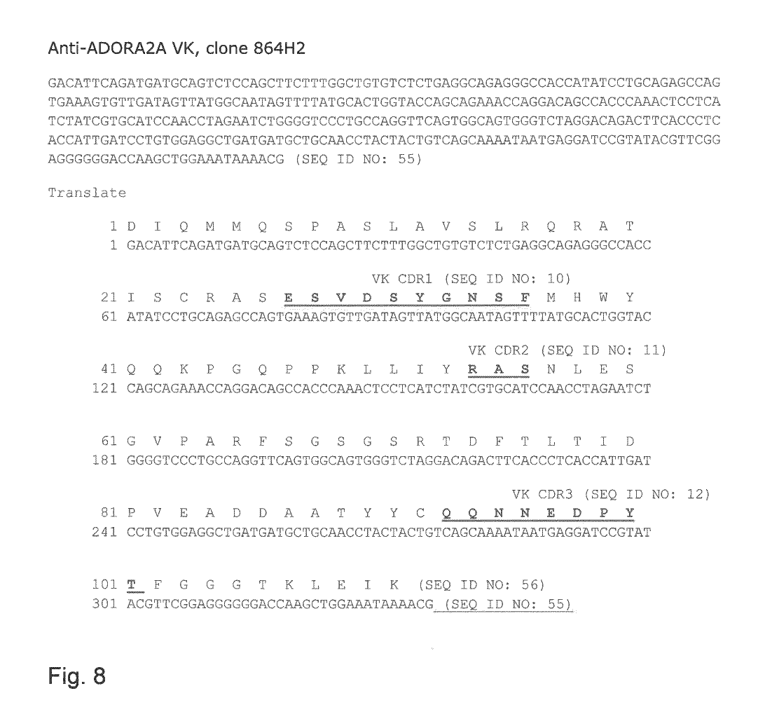

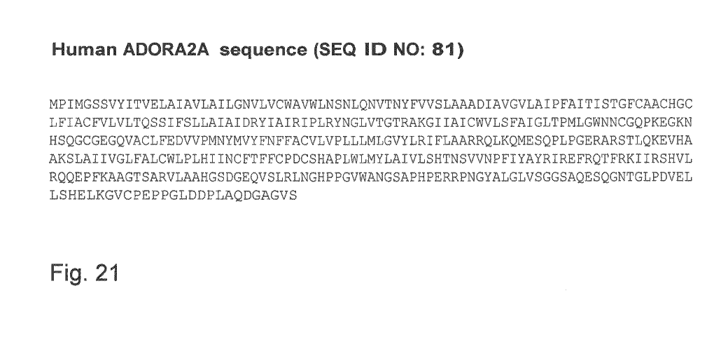

In further aspects, ADORA2A antibodies of the invention comprise any of the full length heavy or light chain sequences provided in FIGS. 5-20. In further embodiments, ADORA2A antibodies of the invention comprise sequences with a sequence identity of about 70%, 75%, 80%, 85%, 90%, 91%, 92%, 93%, 94%, 95%, 96%, 97%, 98%, 99% and 100% identity to any of the full length heavy or light chain sequences provided in FIGS. 8-33.

In some embodiments, ADORA2A antibodies comprise a heavy chain CDR1 with the following consensus sequence: G-X1-TFTSYW, wherein X1 is either Y or N. In some embodiments, ADORA2A antibodies comprise a heavy chain CDR2 with the following consensus sequence: INP-X1-NGG-X2, wherein X1 is N, S, or F and X2 is T or I.

In some embodiments, ADORA2A antibodies comprise a light chain CDR2 with the following consensus sequence: X1-AS, where X1 is G, R, or S.

In further aspects, the present invention provides an expression vector encoding an antibody or protein according to any of the sequences described herein and in accordance with any of the sequences provided in FIGS. 3 and 5-20.

In still further aspects, the present invention provides a method of making an antibody or protein according to any of the sequences described herein and in accordance with any of the sequences provided in FIGS. 3 and 5-20, the method comprising providing a cell comprising a nucleic acid encoding that antibody or protein, where the cell is cultured under conditions suitable for expression of the antibody or protein.

In still further aspects, the present invention provides a method of treating a ADORA2A-associated disease, the method comprising treating a subject in need thereof with an antibody or protein according to any of the sequences described herein and in accordance with any of the sequences provided in FIGS. 3 and 5-20.

In yet further aspects, the antibodies of the invention find use in a variety of applications, including diagnosis of ADORA2A-related diseases and treatment thereof.

### IV. ADDITIONAL MODIFICATIONS TO ADORA2A ANTIBODIES

In addition to any of the modifications and variants outlined above, other modifications can be made. For example, the molecules may be stabilized by the incorporation of disulphide bridges linking the VH and VL domains (Reiter et al., 1996, Nature Biotech. 14:1239-1245, entirely incorporated by reference for all purposes and in particular for all teachings regarding modifications of molecules, including antibodies). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-β-(5-imidozoyl)propionic acid, chloroacety I phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cynomolgusogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention).

Glycosylation

Another type of covalent modification is alterations in glycosylation. In another embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function.

Engineered glycoforms may be generated by a variety of methods known in the art (Umañ a et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zürich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody (e.g. post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

### V. EXPERIMENTAL EXAMPLES

### Example 1

### Development of Antibodies Against Human ADORA2A

Mice were immunized and hybridomas were generated from a single group of animals (fusion 864H). The resulting hybridomas were screened by flow cytometry on ADORA2A expressing, and control cells, to identify those producing specific antibodies against ADORA2A. This resulted in a total of 8 antibodies that react with the extracellular regions of human ADORA2A.

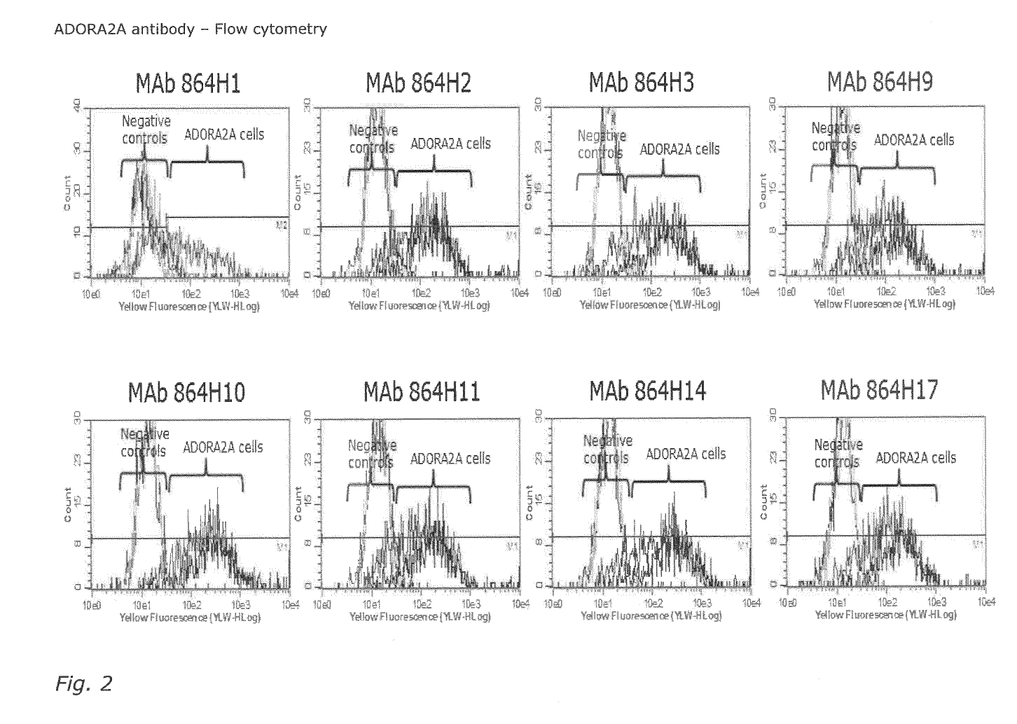

The antibodies produced from each of the hybridomas were used to stain HEK293 cells transfected with ADORA2A and analyzed by flow cytometry. HEK293 cells were transfected with a plasmid expressing ADORA2A either with or without an HA tag (red and black lines in FIG. 2). As negative controls the same cells were stained just with the labeled secondary antibody, or HEK293 cells transfected with an unrelated membrane protein (CD2O). All of the antibodies exhibited significant staining of the ADORA2A transfected cells with 10-100× staining over the negative control cells (FIG. 2).

The heavy and light chain antibody genes from the hybridomas were isolated by RT PCR, cloned, and sequenced. The PCR primers were designed to the beginning and end of the V domains of the heavy and light chains and therefore nucleotide changes in the determined sequence can occur within these regions due to cross priming. However, these are outside the functional CDR sites of the antibody genes that are responsible for binding to the antigen. The sequence was translated, the derivative germline VH and VK genes identified, and the CDR regions identified using the IMGT rules (FIG. 3). All 8 antibodies showed different sequences.

### Example 2

### Comparison to Commercially Available Antibodies

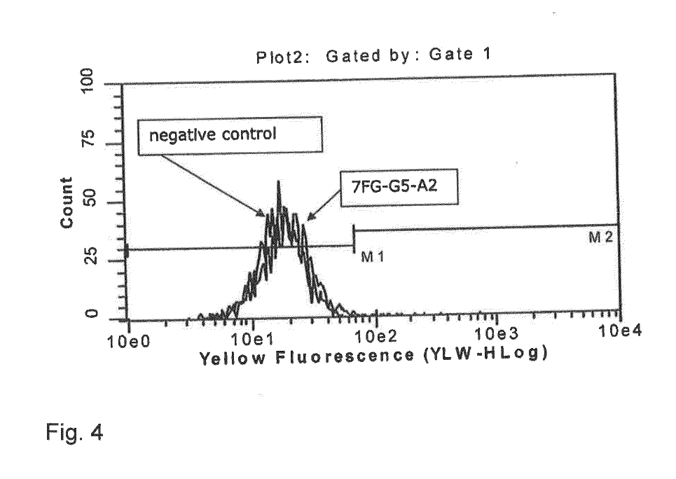

While a number of commercial vendors (including but not limited to Abcam, Acris, Cayman Lifespan, Merck Milipore, USB) list a monoclonal available to ADORA2A all of these antibodies are reported to bind to the sequence SQPLPGER at position 213-220 in the 3^{rd} cytoplasmic loop. From the available specifications and often times identical datasheets it is likely these antibodies are in all the same clone 7FG-G5-A2 (Piersen, C E, et al, Mol. Pharmacol. 45(5) 861-870 (1994)). While listed for flow cytometry it should be apparent that fixation techniques would be required to use this antibody. 7FG-G5-A2, which is reported to react with the aforementioned sequence, shows no reactivity with live HEK293 transfected with ADORA2A as shown in FIG. 4 (7FG-GF-A2 trace and negative control trace identified by arrows), as would be expected for an antibody reactive with cytoplasmic regions of ADORA2A or any GPCR or any transmembrane protein. All other antibodies for ADORA2a in the public domain are polyclonal and listed for use in Western blot or immunohistochemistry, but not flow cytometry and would as such not be expected to react with the intact extracellular portions of ADORA2A.

Each and every reference herein is incorporated by reference in its entirety for all purposes and in particular for any teachings relevant to any of the embodiments discussed herein.

It will be appreciated by those skilled in the art that changes could be made to the exemplary embodiments described above without departing from the broad inventive concept thereof. It is understood, therefore, that this invention is not limited to the exemplary embodiments described, but it is intended to cover modifications within the spirit and scope of the present invention as defined by the claims. For example, specific features of the exemplary embodiments may or may not be part of the claimed invention and features of the disclosed embodiments may be combined.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

1-35. (canceled)

36. An antibody which is:
(a) an antibody comprising a variable heavy chain comprising a first CDR of SEQ ID NO: 1, a second CDR of SEQ ID NO: 2, and a third CDR of SEQ ID NO: 3, or a variant thereof, wherein said variant has from one to four amino acid changes in said first, second or third CDR;
(b) an antibody comprising a variable light chain comprising a first CDR of SEQ ID NO: 4, a second CDR of SEQ ID NO: 5, and a third CDR of SEQ ID NO: 6, or a variant thereof, wherein said variant has from one to three amino acid changes in said first, second or third CDR;
(c) an antibody comprising a variable heavy chain comprising a first CDR of SEQ ID NO: 7, a second CDR of SEQ ID NO: 8, and a third CDR of SEQ ID NO: 9, or a variant thereof, wherein said variant has from one to four amino acid changes in said first, second or third CDR;
(d) an antibody comprising a variable light chain comprising a first CDR of SEQ ID NO: 10, a second CDR of SEQ ID NO: 11, and a third CDR of SEQ ID NO: 12, or a variant thereof, wherein said variant has from one to four amino acid changes in said first, second or third CDR;
(e) an antibody comprising a variable heavy chain comprising a first CDR of SEQ ID NO: 13, a second CDR of SEQ ID NO: 14, and a third CDR of SEQ ID NO: 15, or a variant thereof, wherein said variant has from one to four amino acid changes in said first, second or third CDR;
(f) an antibody comprising a variable light chain comprising a first CDR of SEQ ID NO: 16, a second CDR of SEQ ID NO: 17, and a third CDR of SEQ ID NO: 18, or a variant thereof, wherein said variant has from one to three amino acid changes in said first, second or third CDR;
(g) an antibody comprising a variable heavy chain comprising a first CDR of SEQ ID NO: 19, a second CDR of SEQ ID NO: 20, and a third CDR of SEQ ID NO: 21, or a variant thereof, wherein said variant has from one to four amino acid changes in said first, second or third CDR;
(h) an antibody comprising a variable light chain comprising a first CDR of SEQ ID NO: 22, a second CDR of SEQ ID NO: 23, and a third CDR of SEQ ID NO: 24, or a variant thereof, wherein said variant has from one to three amino acid changes in said first, second or third CDR;
(i) an antibody comprising a variable heavy chain comprising a first CDR of SEQ ID NO: 25, a second CDR of SEQ ID NO: 26, and a third CDR of SEQ ID NO: 27, or a variant thereof, wherein said variant has from one to four amino acid changes in said first, second or third CDR;
(j) an antibody comprising a variable light chain comprising a first CDR of SEQ ID NO: 28, a second CDR of SEQ ID NO: 29, and a third CDR of SEQ ID NO: 30, or a variant thereof, wherein said variant has from one to three amino acid changes in said first, second or third CDR;
(k) an antibody comprising a variable heavy chain comprising a first CDR of SEQ ID NO: 31, a second CDR of SEQ ID NO: 32, and a third CDR of SEQ ID NO: 33, or a variant thereof, wherein said variant has from one to four amino acid changes in said first, second or third CDR;
(l) an antibody comprising a variable light chain comprising a first CDR of SEQ ID NO: 34, a second CDR of SEQ ID NO: 35, and a third CDR of SEQ ID NO: 36, or a variant thereof, wherein said variant has from one to three amino acid changes in said first, second or third CDR;
(m) an antibody comprising a variable heavy chain comprising a first CDR of SEQ ID NO: 37, a second CDR of SEQ ID NO: 38, and a third CDR of SEQ ID NO: 39, or a variant thereof, wherein said variant has from one to four amino acid changes in said first, second or third CDR;
(n) an antibody comprising a variable light chain comprising a first CDR of SEQ ID NO: 40, a second CDR of SEQ ID NO: 41, and a third CDR of SEQ ID NO: 42, or a variant thereof, wherein said variant has from one to three amino acid changes in said first, second or third CDR;
(o) an antibody comprising a variable heavy chain comprising a first CDR of SEQ ID NO: 43, a second CDR of SEQ ID NO: 44, and a third CDR of SEQ ID NO: 45, or a variant thereof, wherein said variant has from one to four amino acid changes in said first, second or third CDR; or
(p) an antibody comprising a variable light chain comprising a first CDR of SEQ ID NO: 46, a second CDR of SEQ ID NO: 47, and a third CDR of SEQ ID NO: 48, or a variant thereof, wherein said variant has from one to three amino acid changes in said first, second or third CDR;
wherein each of said antibodies of (a) to (p) binds human ADORA2A.

37. An antibody according to claim 36, which is
(a) an antibody comprising a first heavy chain CDR comprising SEQ ID NO: 1; a second heavy chain CDR comprising SEQ ID NO: 2; a third heavy chain CDR comprising SEQ ID NO: 3; a first light chain CDR comprising SEQ ID NO: 4; a second light chain CDR comprising SEQ ID NO: 5; and a third light chain CDR comprising SEQ ID NO: 6;
(b) an antibody comprising: a first heavy chain CDR comprising SEQ ID NO: 7; a second heavy chain CDR comprising SEQ ID NO: 8; a third heavy chain CDR comprising SEQ ID NO: 9; a first light chain CDR comprising SEQ ID NO: 10; a second light chain CDR comprising SEQ ID NO: 11; and a third light chain CDR comprising SEQ ID NO: 12;
(c) an antibody comprising: a first heavy chain CDR comprising SEQ ID NO: 13; a second heavy chain CDR comprising SEQ ID NO: 14; a third heavy chain CDR comprising SEQ ID NO: 15; a first light chain CDR comprising SEQ ID NO: 16; a second light chain CDR comprising SEQ ID NO: 17; and a third light chain CDR comprising SEQ ID NO: 18;
(d) an antibody comprising: a first heavy chain CDR comprising SEQ ID NO: 19; a second heavy chain CDR comprising SEQ ID NO: 20; a third heavy chain CDR comprising SEQ ID NO: 21; a first light chain CDR comprising SEQ ID NO: 22; a second light chain CDR comprising SEQ ID NO: 23; and a third light chain CDR comprising SEQ ID NO: 24;
(e) an antibody comprising: a first heavy chain CDR comprising SEQ ID NO: 25; a second heavy chain CDR comprising SEQ ID NO: 26; a third heavy chain CDR comprising SEQ ID NO: 27; a first light chain CDR comprising SEQ ID NO: 28; a second light chain CDR comprising SEQ ID NO: 29; and a third light chain CDR comprising SEQ ID NO: 30;
(f) an antibody comprising: a first heavy chain CDR comprising SEQ ID NO: 31; a second heavy chain CDR comprising SEQ ID NO: 32; a third heavy chain CDR comprising SEQ ID NO: 33; a first light chain CDR comprising SEQ ID NO: 34; a second light chain CDR comprising SEQ ID NO: 35; and a third light chain CDR comprising SEQ ID NO: 36;
(g) n antibody comprising: a first heavy chain CDR comprising SEQ ID NO: 37; a second heavy chain CDR comprising SEQ ID NO: 38; a third heavy chain CDR comprising SEQ ID NO: 39; a first light chain CDR comprising SEQ ID NO: 40; a second light chain CDR comprising SEQ ID NO: 41; and a third light chain CDR comprising SEQ ID NO: 42; or
(h) an antibody comprising: a first heavy chain CDR comprising SEQ ID NO: 43; a second heavy chain CDR comprising SEQ ID NO: 44; a third heavy chain CDR comprising SEQ ID NO: 45; a first light chain CDR comprising SEQ ID NO: 46; a second light chain CDR comprising SEQ ID NO: 47; and a third light chain CDR comprising SEQ ID NO: 48;
wherein each of said antibodies of (a) to (h) binds human ADORA2A.

38. An antibody which is
(a) an antibody comprising a heavy chain variable region comprising any one of SEQ ID NOs: 50, 54, 58, 62, 66, 70, 74, 78;
(b) an antibody comprising a heavy chain variable region having a sequence with at least about 95% sequence identity to any one of SEQ ID NOs: 50, 54, 58, 62, 66, 70, 74, 78, wherein each of said antibody binds human ADORA2A;
(c) an antibody comprising a light chain variable region comprising any one of SEQ ID NOs: 52, 56, 60, 64, 68, 72, 76, 80; or
(d) an antibody comprising a light chain variable region having a sequence with at least about 95% sequence identity to any one of SEQ ID NOs: 52, 56, 60, 64, 68, 72, 76, 80, wherein each of said antibody binds human ADORA2A.

39. An antibody according to claim 8, which is
(a) an antibody comprising a heavy chain variable region comprising any one of SEQ ID NOs: 50, 54, 58, 62, 66, 70, 74, 78; or
(b) an antibody comprising a light chain variable region comprising any one of SEQ ID NOs: 52, 56, 60, 64, 68, 72, 76, 80.

40. An antibody according to claim 36, which is a humanized antibody.

41. An antibody according to claim 37, which is a humanized antibody.

42. An antibody according to claim 38, which is a humanized antibody.

43. An antibody according to claim 39, which is a humanized antibody.

44. A protein which is
(a) a protein comprising a variable heavy chain having any one of SEQ ID NOs: 50, 54, 58, 62, 66, 70, 74, 78; or
(b) a protein comprising a variable light chain having any one of SEQ ID NOs: 52, 56, 60, 64, 68, 72, 76, 80.

45. A nucleic acid encoding an antibody of claim 36.

46. A nucleic acid encoding an antibody of claim 38.

47. A nucleic acid encoding a protein of claim 44.

48. An expression vector which encodes an antibody of claim 36.

49. An expression vector which encodes an antibody of claim 38.

50. An expression vector which encodes a protein of claim 44.

51. A method of making an antibody according to claim 36, comprising culturing a cell comprising a nucleic acid encoding said antibody under conditions suitable for expression of said antibody.

52. A method of making an antibody according to claim 38, comprising culturing a cell comprising a nucleic acid encoding said antibody under conditions suitable for expression of said antibody.

53. A method of making a protein according to claim 44, comprising culturing a cell comprising a nucleic acid encoding said protein under conditions suitable for expression of said protein.

54. A method of treating an ADORA2A-associated disease in a subject in need thereof, comprising administering to said subject, an antibody according to claim 36.

55. A method of treating an ADORA2A-associated disease in a subject in need thereof, comprising administering to said subject, an antibody according to claim 38.

56. A method of treating an ADORA2A-associated disease in a subject in need thereof, comprising administering to said subject, a protein according to claim 44.
